(19) United States
(12) Patent Application Publication
(10) Pub. No.: US 2004/0082063 A1
(43) Pub. Date: Apr. 29, 2004

(54) Tissue-like organization of cells and macroscopic tissue-like constructs, generated by macromass culture of cells, and the method of macromass culture

(75) Inventors: Manisha Sharadchandra Deshpande, Maharashtra (IN); Manoj Vinoy Mojamdar, Maharashtra (IN)

Correspondence Address: LACKENBACH SIEGEL, ONE CHASE ROAD, SCARSDALE, NY, 10583, US

(73) Assignee: Reliance Life Sciences Pvt. Ltd., Mumbai (IN)

(21) Appl. No.: 10/686,822

(22) Filed: Oct. 16, 2003

(30) Foreign Application Priority Data
Oct. 18, 2002 (IN) 912/MUM/2002

(51) Int. Cl. C12N 5/08

(52) U.S. Cl. 435/366

(57) ABSTRACT

Three-dimensional tissue-like organization of cells by high cell-seeding-density culture termed as macromass culture is described. By macromass culture, cells can be made to organize themselves into a tissue-like form without the aid of a scaffold and three-dimensional macroscopic tissue-like constructs can be made wholly from cells. Tissue-like organization and macroscopic tissue-like constructs can be generated from fibroblastic cells of mesenchymal origin (at least), which can be either differentiated cells or multipotent adult stem cells. In this work, tissue-like organization and macroscopic tissue-like constructs have been generated from dermal fibroblasts, adipose stromal cells-derived osteogenic cells, chondrocytes, and from osteoblasts. The factor causing macroscopic tissue formation is large scale culture at high cell seeding density per unit area or three-dimensional space, that is, macromass culture done on a large scale. No scaffold or extraneous matrix is used for tissue generation, the tissues are of completely cellular origin. No other agents (except high cell-seeding-density) that aid in tissue formation such as tissue-inducing chemicals, tissue-inducing growth factors, substratum with special properties, rotational culture, etc, are employed for tissue formation. These tissue-like masses have the potential for use as tissue replacements in the human body. Tissue-like organization by high cell-seeding-density macromass culture can also be generated at the microscopic level.

### FIELD OF THE INVENTION

The present invention relates to tissue engineering. More specifically, this invention relates to generation of three-dimensional tissue-like organization of cells. Further more specifically, this invention relates to the fabrication of three-dimensional macroscopic tissue-like constructs for possible implantation in the human body as a therapy for diseased or damaged conditions.

### BACKGROUND OF THE INVENTION

The human body can be afflicted by several diseased or damaged conditions of different organs, for which one therapeutic approach is the replacement of damaged parts, by extraneously obtained or developed tissue equivalents. For instance, burns or ulcers of the skin can be treated with application of suitable skin equivalents, non-uniting gaps in fractured bone could be treated by implantation of suitable bone substitutes, and damage to articular cartilage could be repaired by suitable cartilage-forming implants.

Every year, surgeons perform surgical procedures to treat patients who experience organ failure or tissue loss. Surgeons/physicians could treat these patients by transplanting organs from one individual to another, performing reconstructive surgery, or by using mechanical devices such as kidney dialyzers, prosthetic hip joints, or mechanical heart valves. Although these approaches have saved many lives, they are subject to limitations. The limitation of transplantation of organs such as the heart, liver, and kidney is not the surgical technique, but the scarce availability of donor organs.

The possible kinds of naturally available implants have been xenografts obtained from animals, allografts obtained from human donors, and autografts obtained from healthy parts of the patient itself. Xenografts have the problem of immunological non-compatibility and transmission of zoonotic pathogens including retroviruses. Allografts have the problem of immune rejection and non-availability of donors. Autografts have the problem of lack of required amount of suitable tissue and increase in trauma to the patient.

For surgical reconstruction, tissue may be moved from one part of the patient to another part. These autografts (tissue grafts from the patient) include skin grafts for burns, blood vessel grafts for heart bypass surgeries, and nerve grafts for facial and hand reconstruction. The disadvantages of using autografts also include the need for multiple surgeries and loss of function at the donor site. In addition, surgical reconstruction often involves using the body's tissues for purposes not originally intended and can result in long-term complications.

As a result of these drawbacks of existing therapeutic options, there is a requirement for engineered tissue equivalents, and what has emerged as a new discipline is the science of tissue engineering. Its goals are to create tissues in culture for use not only as model systems in fundamental studies, but more importantly, for use as replacement tissues for damaged or diseased body parts. Although, efforts to generate bioartificial tissues and organs for human therapies go back at least thirty years, such efforts have come closer to clinical success only in the last ten years. This has been made possible by major advances in molecular and cell biology, cell culture technologies, and materials science.

The term “tissue engineering” is relatively recent and has been used more widely in the last five years to describe the interdisciplinary field that applies the principles of engineering and the life sciences toward the development of bioartificial tissues and organs.

One of the major strategies adopted for the creation of lab-grown tissues is the growth of isolated cells on three-dimensional templates or scaffolds (matrices) under conditions that will coax the cells to develop into a functional tissue. When implanted, this bioartificial tissue should become structurally and functionally integrated into the body. The matrices can be fashioned from natural materials such as collagen or from synthetic polymers such as plastics. Ultimately, the scaffold material should be biodegradable over time and should serve as an initial three-dimensional template for tissue growth.

As the cells grow and differentiate on the scaffold, they will produce various proteins needed to recreate a tissue. Degradation of the scaffold ensures that only natural tissue remains in the body. There are also different kinds of bioreactors incorporating different technologies for the task of building a tissue from cells.

Virtually every tissue in the body is a potential target for bioengineering and progress is occurring rapidly on many fronts. For the skin as an organ, different kinds of engineered replacements have been developed—skin has been re-engineered using several different approaches with varying degrees of success.

U.S. Pat. No. 5,489,304 describes a non-cellular graft which has a synthetic outer layer bonded to a collagen-chondroitin sulfate-derived dermal analog layer. This replacement, which is placed initially on the wound before a cultured epithelial autograft is applied, has the disadvantage that it lacks the growth factors important for skin wound healing or the cells that can supply these factors.

U.S. Pat. No. 5,460,939 describes another graft, which is cellular. Here, fibroblasts are grown in bio-resorbable lactic acid/glycolic acid copolymer mesh to form a sheet. In this graft, the scaffolding mesh is not of natural origin.

Eaglstein & Falanga (1997) describe a skin graft, which includes a dermal layer having fibroblasts grown in a bovine collagen matrix. In this graft, extracellular matrix is provided extraneously to the cells, which although manufacture human collagen, but, the extraneous component remains at the time of graft application.

U.S. Pat. No. 5,613,982 describes a graft, in which human cadaver skin is processed to remove antigenic cellular components, leaving an immunologically inert dermal layer. This has the limitation of being acellular and of non-availability of human cadaver skin easily.

In all of the above examples, the technological requirements for production of the equivalents are fairly complex, hence would add to the cost of the product. Cellular sheets of fibroblasts using ascorbate have been developed, but the formation of such sheets requires about 35 days (Michel et al, 1999; L'Heureux et al, 1998).

Thus, there exists a need for the development of a dermal equivalent, the materials for which are easily available, which has no synthetic or natural extraneous matrix that could cause an inflammatory reaction in some patients, which is cellular so that it can produce growth factors and other proteins, which can be prepared in a relatively shorter time, and the preparation of which is technologically simple so that the product is more cost-effective.

An area that requires attention in the field of tissue-engineered products is bone substitutes for patients whose fractures do not heal, leaving non-uniting gaps. Autologous bone grafting increases the trauma to the patient. Different approaches are being tried in bone engineering (Service, 2000). Biomaterials such as collagen matrix infused with growth factors that trigger bone formation have been tried, but such constructs lack the cellular component and the incorporation of the required substantial amount of growth factors makes it a very expensive alternative. Ceramic or hydroxyapatite matrices seeded with mesenchymal stem cells are other approaches, but the use of such scaffolds may not be ideal for the human body. Thus, there exists a need for cost-effective cellular implants which would cause the healing of bone.

Another area that requires attention in the field of tissue-engineered products is cartilage repair. It is a known fact that articular cartilage has limited capacity for complete repair after injury. The cell-based therapy of autologous chondrocyte implantation has shown good clinical results (McPherson & Tubo, 2000) but there remains ample scope for improvement because, the time for complete repair is very long. Possibly, a pre-formed tissue rather than cell suspension would give better results upon implantation. Also, a preformed tissue has an advantage over free cells for surgical implantation. Therefore, various approaches are being tried in making a cartilage-like construct using cells and scaffold, but an ideal scaffolding matrix that will allow the cells in the implant to closely mimic the natural cartilage formation process remains a challenge (Kim & Han, 2000). Thus, there exists a need for developing a preformed tissue that could efficiently initiate cartilage repair when implanted at the site of injury, and which would also be cost-effective.

To summarize, there is a requirement for developing relatively inexpensive living cellular tissue substitutes for therapeutic purposes. The technologically complex bioreactors mentioned earlier for developing three-dimesional tissues are expensive methodologies. Also, in general, there is always a need for the development of tissue substitutes by new methods, which when tested, could prove to have better performance in one or more respects than existing replacements.

Looking to the need of the hour, the scientists of the present invention, have developed novel three-dimensional macroscopic tissue-like constructs which have potential to be used as tissue replacements in human body. A novel characteristic of these tissue-like constructs is that, no scaffold or extraneous matrix is required for tissue generation, the tissues can be formed of completely cellular origin. Also, no other agents that aid in tissue formation (except high cell-seeding-density) such as tissue-inducing chemicals, tissue-inducing growth factors, substratum with special properties, rotational culture are employed for tissue formation. There are no specific complex medium requirements for tissue-like construct formation. The factor causing macroscopic tissue-like construct formation is, large scale culture of cells at high cell seeding per unit area or space.

A crucial aspect of tissue engineering is how to make cells assemble into a tissue or three-dimensional structure. The present invention gives a novel method to achieve the same.

### OBJECTS OF THE INVENTION

i. In the light of the above, it is therefore an object of the present invention to provide a novel method of assembling cells into three-dimensional tissue-like organization and tissue-like constructs.

ii. Also in the light of the above, it is therefore an object of the present invention to provide three-dimensional macroscopic tissue like constructs for possible implantation in the human body as a therapy for diseased or damaged conditions.

iii. It is another object of the present invention to provide macroscopic tissue-like constructs that are histologically competent. By “histological competence” it is meant that these tissue-like constructs can be sectioned easily without disruption.

iv. It is still another object of the present invention to provide three-dimensional tissue-like organization of cells and cost-effective putative tissue equivalents made from fibroblastic cells of mesenchymal origin (at least), such as an engineered putative dermal equivalent made from dermal fibroblasts, putative substitute with bone-like properties made from adipose stromal cells-derived osteogenic cells or from osteoblasts and putative substitute for cartilage repair made from chondrocytes. It is a related object of the present invention to bring forth the possibility of providing other tissues also, which are possible to be constructed from the corresponding cell types by the method of the present invention, if these other cell types have the properties enabling them to undergo tissue-like mass formation upon macromass culture as defined in this invention.

v. It is still another object of the present invention to provide three-dimensional tissue-like organization of cells and macroscopic tissue like constructs without using scaffold or extraneous matrix or complex bioreactor for tissue generation.

vi. It is still another object of the present invention to provide three-dimensional tissue-like organization of cells and macroscopic tissue like constructs without using any agents that aid in tissue formation such as tissue-inducing chemicals, tissue-inducing growth factors, substratum with special properties, rotational culture, etc.

vii. It is still another object of the present invention to provide three-dimensional tissue-like organization of cells and macroscopic tissue-like constructs of different kinds, formed by using high cell seeding density per unit area or space of culture vessel, without requirement for any other agent that aids in tissue formation.

viii. It is yet another object of the present invention to provide macroscopic tissue-like constructs which have a high cell density in the final form.

ix. It is yet another object of the present invention to provide tissue-like organization of cells and macroscopic tissue-like constructs which can be formed without the requirement of specific complex medium components.

x. It is yet another object of the present invention to provide tissue-like organization of cells and macroscopic tissue-like constructs the properties of which can be modulated to include desired properties by suitable change/s in the growth and/or tissue-forming medium.

xi. It is yet another object of the present invention to provide tissue-like organization of cells and macroscopic tissue-like constructs, which can be formed by macromass culture on different compatible growth surfaces according to requirement.

xii. It is yet another object of the present invention to provide macroscopic tissue-like constructs which can be scaled-up to larger sizes by simple scaling-up in two dimensions of the method used for their formation, viz., macromass culture.

xiii. Another object of this invention is to produce three-dimensional tissue-like organization at the microscopic level.

### DESCRIPTION OF THE INVENTION

In the present invention, there is provided a method for the assembly of cells into three-dimensional tissue-like organization by macromass culture, and the novel method of macromass culture. There are provided macroscopic three-dimensional tissue-like constructs that are histologically competent, generated by macromass culture of cells. The present invention relates to tissue engineering. More specifically, this invention relates to fabrication of three-dimensional tissue like constructs for possible implantation in the human body as a therapy for diseased or damaged conditions. This invention gives a method for the organization of cells into three-dimensional tissue-like forms and describes the tissue-like forms themselves.

Fabrication of tissues is a goal important for the replacement of diseased tissues in the human body. Efforts are being made to explore and recruit the tissue-forming abilities of cells for tissue engineering.

The process of tissue engineering of cellular grafts involves the following two (2) major steps

i. procuring the cells from suitable sources. The procured cells could require suitable preparation such as differentiation into the desired cell type.

ii. constructing the tissue using suitably prepared cells to produce different tissue engineered products.

The present invention addresses the second of these steps. The inventors have developed a simple and cost effective method for the generation of three-dimensional tissue-like organization of cells and formation of living, cellular, putative tissue substitutes.

The tissue-like constructs of the present invention have the cohesive strength to be able to withstand physical manipulation and handling as would be required for the procedure of placing them surgically at the required site in the body from the container holding them, with the aid of appropriate supporting and handling devices or instruments.

Substantial amount of work has been done till date, in the generation of tissue substitutes that are scaffold-based—these include a scaffold as an important structural and often functional component. This scaffold requires to have properties of biocompatibility, biodegradability (so that eventually only natural tissue remains in the body) and of providing a permissive environment for optimal cellular function. The development of scaffolds that are ideal in all possible respects remains a challenge. The present invention has the advantage that it circumvents the need to incorporate a scaffold because the three-dimensional tissue-like constructs generated by the present invention are made without the aid of a scaffold. Formation of histologically competent tissue-like constructs by the macromass method of the present invention does not require a scaffold. Thus the tissue-like constructs of the present invention also eliminate any adverse effects or drawbacks that could be associated with the use of a scaffold which is less than ideal in any respect. In the present invention, extracellular matrix is synthesized by the cells themselves, there are no extraneous matrix components used. Tissue formation takes place simply by seeding the cells at a high cell density per unit area or space of culture vessel. This has been termed as “macromass” culture which is defined as a culture system for three-dimensional tissue-like formation or organization of cells, in which, cells are seeded at a high density per unit area or space of a culture vessel in a range spanning a window around 10⁶ cells per cm² and there is no requirement for any other agents that aid in tissue formation. A broader definition of macromass culture is a method of generating three-dimensional tissue-like organization, macroscopic or microscopic, from cells by high-density cell seeding, bringing cells together in close proximity in a certain favorable range of high densities of cells in three-dimensional space, that favors cohesive integration of cells into a three-dimensional tissue-like state, there being no requirement for any other agents that aid in tissue formation. A certain high seeding density of cells within a favorable range is required to be achieved within a given space. In the macromass range of favorable high cell seeding densities, when the cells are settled together within the three-dimensional space that is occupied by the cells at the base of the culture vessel, they come into a state of close proximity with one another that triggers or signals them into a tissue formation mode by which they become cohesively integrated. (It may be noted that the macromass range of cell seeding density could be achieved in a vessel with a flat or curved base) The result of using a culture vessel about 0.75 cm in diameter or larger for macromass culture is the formation of macroscopic three-dimensional tissue-like constructs, wherein “macroscopic” means that the size of the tissue is at least such that it can be easily visually discerned by the normal unaided human eye.

A previously known tissue culture system, high-density micromass culture has been used for the chondrogenic differentiation of cells, and the scale of such culture has been limited to being 10 to 20 μl spots of cell suspension (Yoon et al, 2000). Classically, limb mesenchymal cells when cultured in vitro as micromass cultures, undergo formation of precartilage condensations or aggregates which are present as individual nodules covering the area of the micromass spot (Ahrens et al, 1977). The cell nodules thus formed are separate from one another with cells not formed into nodules in between and are microscopic. The larger, yet microscopic, spheroidal structures in which all the cells come together to form one aggregate are generated with the requirement of specific components added to the culture medium such as growth factors, as mentioned later in the text. However, the tissue-like masses generated by the present inventors are macroscopic (and formed without the aid of any specific agent that aids in tissue formation), and thus possess the desirable quality of size required to have potential as tissue replacements for the human body. In the tissue-like organization by macromass culture of the present invention, all cells become part of the integrated tissue-like organization which is thus whole; there are no individual nodules. It has been earlier found that, by micromass culture, leg precartilage mesenchymal cells produced a nodular pattern (Downie & Newman, 1994). While wing precartilage mesenchymal cells produced a sheet pattern by micromass culture; this was in a serum-free culture system, unlike the macromass culture system of the present invention. Also, the leg precartilage mesenchymal cells could produce a sheet-like pattern, but this was upon treatment with TGFβ1 in serum-free medium, again unlike macromass culture, wherein no specific agent that aids in tissue formation is required for tissue-like sheet formation and there is no requirement for serum-free conditions.

Hitherto, the question whether cell-cell aggregation leading to whole tissue-like mass formation will occur by high cell-seeding-density culture without any specific agents that aid in tissue formation in the medium had not been investigated. However, the work of the present inventors has addressed this question and the present invention answers in the affirmative.

High-density culture has been used to induce chondrogenesis with microscopic individual nodule formation, but has not been assessed so far, on the larger macroscopic scale for generation of macroscopic tissues for replacement in the human body. And even on the microscopic scale, as mentioned above, whole aggregates are formed only with the help of specific agents, unlike macromass culture of the present invention.

Although the term “macromass” at first perception may appear to mean a mere extension of “micromass”, it is actually different in the important respect that micromass has been developed as a method for chondrogenic differentiation of cells and also includes specific complex medium requirements for even microscopic whole spheroidal aggregate formation (as mentioned below), while macromass is a method for the generation of three-dimensional tissue-like organization of cells and macroscopic tissue-like constructs, and without specific complex medium requirements for formation.

Till date, efforts have been made towards development of cellular aggregates, the results of which have been microscopic masses termed spheroids. Spheroids are three-dimensional cellular structures that have been made from hepatocytes and other cells with the help of a variety of agents that aid in tissue-like formation like non-adherent dishes (Takezawa et al, 1993), spinner-flask culture (Abu-Absi et al, 2002), polymeric substances like Eudragit (Yamada et al, 1998), Matrigel (Lang et al, 2001), Primaria dishes (Hamamoto et al, 1998), poly-D-Lysine coated dishes (Hamamoto et al, 1998), proteoglycan coating (Shinji et al, 1988), culture medium flow (Pollok et al, 1998), rotational culture (Furukawa et al, 2001), liquid overlay technique (Davies et al, 2002), factors enhancing cell-cell adhesion such as insulin, dexamethasone & fibroblast growth factor (Furukawa et al, 2001), aggregation-promoting polymer-peptide conjugates (Baldwin & Saltzman, 2001), rotating-wall bioreactor (Baldwin & Saltzman, 2001), etc. Unlike these spheroids, the tissue masses made in our work are generated without the aid of any such agent that aids in tissue-like formation. The above mentioned spheroids are much smaller, being mostly in the micrometer or sub-millimeter range. Since it is possible to make macroscopic tissue masses by macromass culture as described in the present invention, these have a clear advantage over spheroids for placement in required locations in the human body.

The largest of spheroids (about 1 mm in diameter) the present inventors have found in published literature was formed by high-density pellet culture (Mackay et al, 1998). Their formation took place in the presence of a serum-free defined medium containing TGF-β3, dexamethasone, ascorbate 2-phosphate and insulin-transferrin-selenium supplement where as such a serum-free defined medium is not required for tissue generation by macromass culture. In the preceding report using pellet culture of bone-marrow derived mesenchymal progenitor cells, it had been found that spheroidal aggregate formation did not take place in the pellets incubated in DMEM+10% FCS (Johnstone et al, 1998), while tissue-like constructs by macromass culture form in DMEM+10% FCS. Spheroid formation by micromass culture of multipotential mesenchymal cells has been reported; here again spheroidal aggregate formation took place only upon treatment of the micromass culture with TGFβ1 or bovine bone extract (Denker et al, 1995). Microscopic bone cell spheroids have been reported to form in the presence of serum-free medium containing TGFβ1 (Kale et al, 2000; U.S. patent application Ser. No. 20020127711) and culture of cells in the absence of serum and in the presence of TGFβ1 is essential for bone cell spheroid formation in this method as reported. In the above work, cell culture densities of about 1×10³ cells/cm² to 1×10⁶ cells/cm² have been described as being favorable for spheroid formation. In addition to the other features of the tissue-like constructs of the present invention that distinguish it from the above work, namely, being macroscopic, not requiring absence of serum & not requiring TGFβ1 for formation, the favorable seeding density range also is different. In our work, tissue-like construct does not occur at 1×10⁵ cells/cm² or below, while 1×10³ cells/cm² of the above work is a 100-fold lower. Cell-aggregates or nodules of osteogenic embryonic stem cells have been formed (Buttery et al, 2001), but again, these nodules were microscopic and not formed by high cell-seeding-density culture as described in the present invention. The tissue masses of the present invention, that can be generated by macromass culture when done on a large scale, are macroscopic, hence magnitudes much larger than any spheroids that have been developed, and have no such specific complex medium requirements for formation. A simple medium such as DMEM+10% FCS suffices for tissue-like construct formation by macromass culture. Cohesive plugs of cells have been earlier formed from chondrogenic cells (U.S. Pat. No. 5,723,331), but a preshaped well having a surface that discourages cell attachment and thus deprives cells of anchorage is a critical requirement for this, whereas, in the present invention, there is no requirement for such a surface for tissue-like construct formation.

In one embodiment of this invention, a tissue-like sheet is formed from human dermal fibroblasts as a potential dermal substitute. The dermal fibroblasts can be of allogeneic origin, since it is known that human dermal fibroblasts are relatively non-immunogenic upon transfer to an allogeneic recipient (U.S. Pat. No. 5,460,939). This tissue-like sheet has the potential to be a dermal equivalent, the materials for which are easily available, which has no synthetic or natural extraneous matrix that could cause an inflammatory reaction in some patients, which is cellular so that it can produce growth factors and other proteins, which can be prepared in a relatively shorter time, and the preparation of which is technologically simple so that the product is more cost-effective.

In another embodiment of this invention, by macromass culture, a tissue-like mass with bone-like properties is generated from adipose stromal cells-derived osteogenic cells, as a putative tissue substitute that could have the potential to cause healing of small non-uniting gaps in bone fractures. This could be a possible autologous therapy. This tissue-like mass could have the potential to be a cost-effective cellular implant, devoid of extraneous scaffold, which could cause the healing of small gaps in bone. In this invention, a tissue-like construct has also been made from bone-derived osteoblasts.

In yet another embodiment of the invention, a tissue-like sheet has been developed from human chondrocytes, as a putative implant inducing cartilage repair in patients with articular cartilage damage. Autologous chondrocytes could be used for this tissue-therapy, since chondrocytes can be obtained from small biopsies of cartilage. This tissue-like sheet could have the potential to be a preformed tissue that could efficiently initiate cartilage repair when implanted at the site of injury, and which would also be cost-effective.

In an additional embodiment of this invention, microscopic three-dimensional tissue-like organization is generated by macromass culture within a gelatin sponge.

To summarize, tissue-like constructs of the present invention, generated by macromass culture could have the potential to be living, cellular, tissue substitutes that are free of scaffolds and of extraneous extracellular matrix, and that are technologically simple to make and hence would be cost-effective. The tissue-like constructs of the present invention, described in detail as tissue substitutes for therapeutic purposes, could also find other applications as well, such as in vitro drug testing and the like.

### BRIEF DESCRIPTION OF THE DRAWINGS

Preferred embodiments of the present invention are further illustrated in the accompanying figures, as described below

FIG. 1. Photographs showing the macroscopic tissue-like constructs formed by macromass culture, in 3.5 cm dishes, from (a) dermal fibroblasts (b) adipose stromal cells (c) chondrocytes (d) osteoblasts.

FIG. 2. Cell-cell adhesion process resulting in tissue-like construct formation taking place in macromass culture of adipose stromal cells (a) One hour after start of macromass culture (b) Six hours after start of macromass culture.

FIG. 3. Histological examination of tissue-sheet formed by macromass culture of dermal fibroblasts (a) Hematoxylin & eosin staining shows the three-dimensional organization (b) Masson-Trichome staining shows collagen synthesis.

FIG. 4. Histological examination of tissue-like construct formed by macromass culture of osteogenic cells derived from adipose stromal cells (a) Hematoxylin & eosin staining shows three-dimensional organization (b) Masson-Trichome staining.

FIG. 5. Histological examination of tissue-like construct formed by macromass culture of chondrocytes (a) Hematoxylin & eosin staining shows three-dimensional organization (b) Masson-Trichome staining.

FIG. 6. Collagen type I immunostaining of histological section of tissue-like construct made from dermal fibroblasts, showing positive detection of collagen type I.

FIG. 7. Cells regrown from dissociated tissue sheet made from dermal fibroblasts by macromass culture for assessing viability.

FIG. 8. Gene expression analysis of tissue sheet formed from dermal fibroblasts by macromass culture, assayed by Reverse Transcriptase-PCR. The RT-PCR products corresponding to various genes known to be important for the wound healing process of the skin are shown electrophoresed on 2% agarose gel (M) DNA molecular size marker (1) Collagen type II (2) Syndecan 2 (3) Tenascin-C (4) Vascular endothelial growth factor (5) Collagen type III (6) Fibronectin (7) Keratinocyte growth factor (8) Transforming growth factor 1β.

FIG. 9. Gene expression in tissue-like construct made from osteogenic cells derived from adipose stromal cells. The RT-PCR products are shown electrophoresed on 2% agarose gel (M) DNA molecular size marker (1) Collagen type I (2) Osteopontin (3) Parathyroid hormone receptor (4) Bone morphogenetic protein 2 (5) Bone morphogenetic protein 4 (6) Bone morphogenetic protein receptor IA (7) Bone morphogenetic protein receptor IB.

FIG. 10. Gene expression in tissue-like construct made from chondrocytes. The RT-PCR products are shown electrophoresed on 2% agarose gel (M) DNA molecular size marker (1) Aggrecan (2) Collagen type II (3) Collagen type X.

FIG. 11. Histological analyses of tissue-like construct made from osteogenic cells derived from adipose stromal cells in the presence of conditioned osteogenic medium showing (a) focal actual bone formation within the tissue-like construct (Masson-Trichome) and (b) focal calcium deposition (Von Kossa), demonstrating that the properties of the tissue-like constructs made by macromass culture can be modulated by changes in the medium.

FIG. 12. Histological sections (Toluidine Blue staining) of tissue-like constructs made from chondrocytes in the presence of (a) DMEM+10% FCS (b) Chondrogenic medium, showing cartilage-specific extracellular matrix formation in (b), demonstrating that the properties of the tissue-like constructs made by macromass culture can be modulated by changes in the medium.

FIG. 13. Histological section (Masson-Trichome staining) of composite object consisting of tissue-like sheet made from dermal fibroblasts and a collagen+fibrin gel, demonstrating that tissue-like organization made by macromass culture can be a component of an object.

FIG. 14. Histological section (Hematoxylin & eosin staining) of macromass culture within a gelatin sponge, showing clusters of microscopic three-dimensional tissue-like organizations formed.

Various other aspects of the invention are described in further details in the following sections.

### MATERIALS AND METHODS

It is clarified by the inventors of the present invention that, throughout the entire description of this invention and in the appended claims, although area of culture vessel has been referred to in terms of diameter of a circular culture vessel, the aspect being described actually includes a culture vessel of any shape, its area being same as that of a circular vessel of the mentioned diameter. Also, macromass culture could be achieved in a culture vessel with a flat or non-flat base, although work presented in this description relates to a vessel with a flat base.

7.1. Cell Isolation, Media and Culture

In the present invention, human dermal fibroblasts were isolated from human skin biopsies. The dermis was separated from the epidermis by treatment with Dispase (Sigma, St. Louis, USA). The dermis was minced and digested with 0.01% collagenase in DMEM+10% FCS overnight and then cells were allowed to attach. Cells were cultured in DMEM+10% FCS at 37° C. in 5% CO₂ and subcultured using Trypsin-EDTA solution. Adipose stromal cells were isolated from human liposuction material according to the protocol described by Zuk et al (2001). These cells were maintained in DMEM+10% FCS. These cells were induced into osteogenic differentiation according to the protocol described by Zuk et al (2001). Chondrocytes were isolated from human cartilage fragment by mincing the cartilage and treating with collagenase before incubating in the maintenance medium of DMEM+10% FCS. Osteoblasts were isolated from human bone by a similar procedure and maintained in DMEM+10% FCS having 50 μg/ml ascorbic acid. Conditioned osteogenic medium was prepared by using the medium in which osteogenic adipose stromal cells were being grown as part of the osteogenic medium for the same cells in the next subculture. Chondrogenic medium was prepared according to Zuk et al (2001).

7.2. Tissue-like Construct Formation

Formation of tissue-like constructs was achieved by macromass culture, which is the novel method of the present invention, earlier defined in the present description of this invention.

Cultured cells were harvested using Trypsin-EDTA. They were resuspended in appropriate volume of medium and seeded preferably in culture dishes with a well diameter of 3.5 cm at a seeding density of about 10⁶ cells per cm² or in the macromass favorable range of tissue-forming cell densities. Thus, preferably, about 10⁷ cells total were seeded in a single well of a six well plate (9.6 cm² area) for the formation of a single tissue. For smaller or larger tissue-like constructs, the total number of cells seeded was adjusted so as to achieve seeding density favorable for tissue-like organization.

7.3. Histological Analyses

Upon formation, tissue-like constructs were fixed and processed for histological examination. Von Kossa staining and Alcian blue staining were done on the appropriate tissue constructs of the present invention, according to the methods described by Zuk et al (2001) and Bancroft et al (1994). Oil Red O staining was done by the method described by Bancroft et al (1994). Toluidine Blue staining was done as follows—After deparaffinization and hydration of sections, they were stained for 1 minute in 2% aqueous Toluidine Blue solution, then washed in water for 2-3 mins. The sections were then dehydrated in 2 changes of 100% acetone, then cleared in xylene and mounted.

Other histological procedures were performed by the Histopathology Laboratory at Sir Hurkisondas Nurrotamdas Hospital & Research Centre, Mumbai, India.

7.4. Immunohistochemistry

Collagen type I immunostaining was performed on sections of paraffin-embedded tissue using goat anti collagen type I antibody and the ABC Staining System of Santa Cruz Biotechnology, Santa Cruz, USA.

7.5. Viability of Cells in Tissue-like Constructs Formed

The tissue masses of the present invention were minced, digested with 0.5 mg/ml collagenase in serum free DMEM for 15 mins, and the released cells were resuspended in growth medium. An aliquot was stained with Trypan Blue. The cells were seeded in a culture flask to assess viability.

7.6. Gene Expression Analysis

Gene expression in the tissue-like constructs made from dermal fibroblasts, osteogenic adipose stromal cells and chondrocytes was analyzed by Reverse Transcriptase-PCR. RNA was extracted from the tissue-like construct using Trizol (Gibco-BRL, Grand Island, USA). RT-PCR was performed using primers specific for the respective genes and the Titan One-Tube RT-PCR system (Roche, Mannheim, Germany).

7.7 Collagen+Fibrin Gel Preparation

For preparation of collagen+fibrin gel, 134 μl of 3.33 mg/ml rat tail collagen type I (Sigma, St. Louis, USA) in 0.1 N acetic acid, 8 μl of 4N NaOH, 165 μl of 28.8 mg/ml fibrinogen in 1× DMEM, and 210 μl of 1.48 mg/ml thrombin in 1.66×DMEM were mixed. The mix was allowed to gel for 2 hours at 37° C.

7.8 Tissue Formation by Macromass Culture within Gelatin Sponge

Gelatin sponge used was AbGel (Sri Gopal Krishna Labs. Pvt. Ltd, Mumbai, India).

### RESULTS AND DISCUSSION

8.1. Formation of Three-dimensional Tissue-like Organization and Macroscopic Tissue-like Constructs

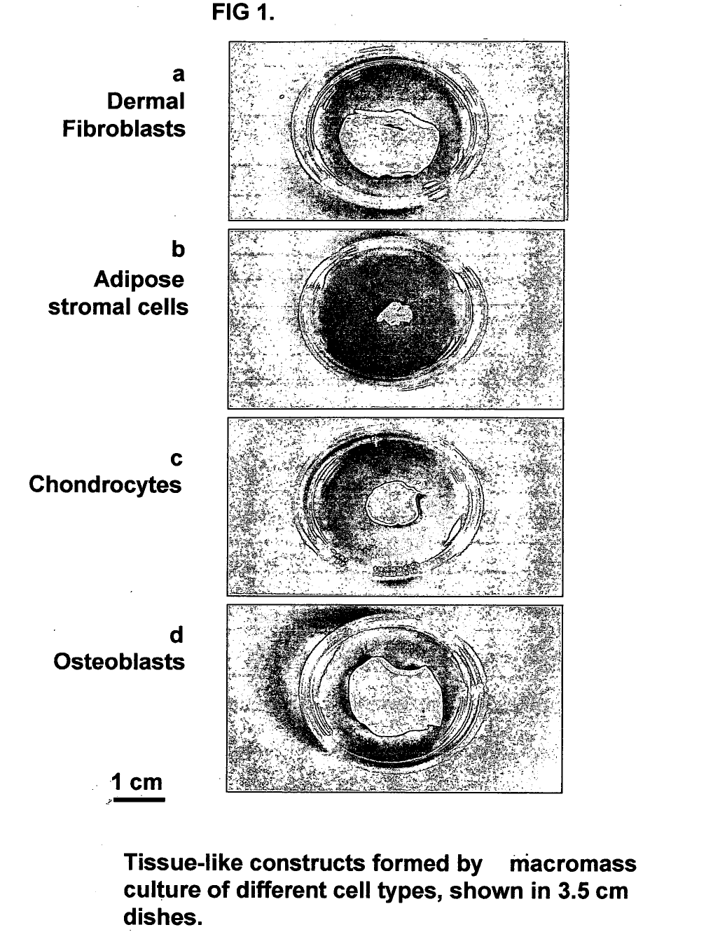

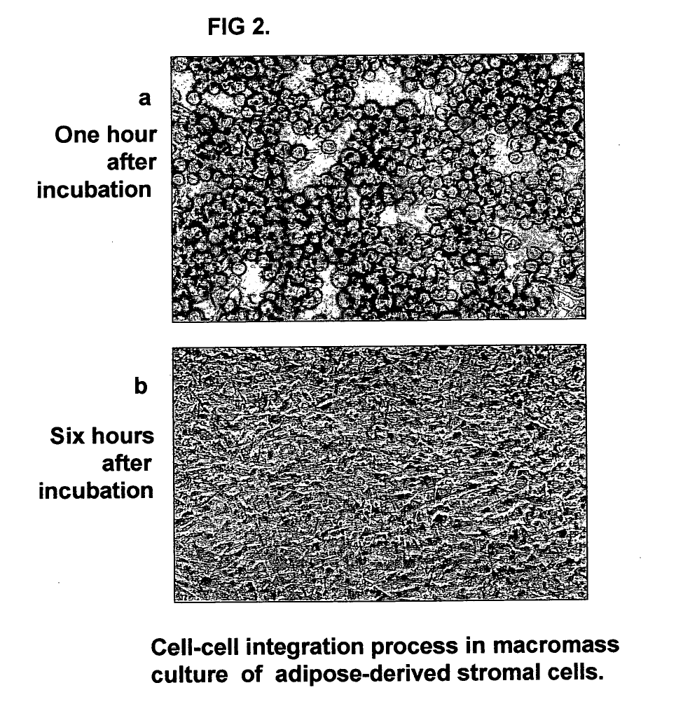

By macromass culture, tissue-like masses (FIG. 1) were formed from dermal fibroblasts in the presence of DMEM+10% FCS in the shape of a sheet which either detached from the growth surface spontaneously or by gentle peeling with a blunt instrument. The adipose stromal cells also formed a tissue-like mass in DMEM+10% FCS which was negative for lipids by Oil Red O staining. Since this was the case, to make possibly useful tissue from adipose stromal cells, they were differentiated into osteogenic cells, which upon macromass culture formed a similar sheet which can contract to a tight mass upon further incubation after detaching. Chondrocytes isolated from human cartilage also formed such a tissue sheet upon macromass culture in DMEM+10%FCS. Osteoblasts isolated from human bone also formed a tissue-like construct by macromass culture in DMEM+10% FCS, after washing away the maintenance medium containing ascorbic acid. Integration of cells appears to be playing an important role in such tissue-like construct formation as seen from the extensive formation of extensions from the cells and cell integration, shown in FIG. 2. When dermal fibroblast macromass culture was scaled up by seeding cells at the mentioned seeding density in a 8.5 cm petri dish, a much larger sheet formed. Thus, it appears that macromass culture can be directly scaled up area wise to obtain as large tissues as desired. Dermal fibroblasts also formed a sheet by macromass culture in serum free DMEM, but the time of formation was greater than in DMEM containing 10% FCS, overnight compared to 3-4 hours in serum-containing medium. In serum-containing medium, the time of formation of tissue from dermal fibroblasts, from adipose stromal cells & osteogenic cells derived from them was about 4 hours, and from chondrocytes was about 18 hours. Osteogenic cells derived from adipose stromal cells formed a tissue-like mass in osteogenic medium as well as in DMEM+10% FCS, after washing the cells to remove osteogenic medium containing ascorbic acid. Generation of tissue-like constructs by macromass culture has been done successfully in culture vessels of diameter 0.75 cm to 8.5 cm. It can be extrapolated that macromass culture in culture vessels smaller than 0.75 cm diameter and larger than 8.5 cm diameter would result in formation of smaller or larger tissue-like constructs respectively. Thus, the dimensions of the three-dimensional tissue-like constructs can be varied.

Although these tissue-like constructs are not fully formed tissues, they could be capable of inducing and participating in the healing process in the body, in a way analogous to the findings that implantation of even stem cells or partially differentiated cells (which are not fully formed tissue but are at the very beginning of tissue formation) can lead to repair and regeneration (Kaji & Leiden, 2001).

It was found that the phenomenon of three-dimensional tissue-like mass formation by macromass culture was dependent on cell seeding density. To examine whether tissue formation took place at all high densities or not, dermal fibroblasts were seeded at a range of different cell densities per unit area. It was found that tangible sheet formation took place at about 3.33×10⁵ cell per cm², while at a seeding density of 6.66×10⁴ cells per cm² (five times lesser) or lower, no tissue sheet formed. Also, tissue sheet formation occurred at the seeding density of 3×10⁶ cells per cm² but not at 7×10⁶ cell per cm² or higher; at the latter seeding density the cells only loosely clumped together but did not form a cohesive tissue mass. Thus, tissue sheet formation took place at 3.33×10⁵ cell per cm² and at 3×10⁶ cells per cm² as well as all densities lying between these two figures that were tested. At the densities tested above or below this range, tissue formation did not occur. Similar experiments were done with native adipose stromal cells and with chondrocytes and the results are tabulated in Table 1. As with dermal fibroblasts, there was a minimum and maximum tissue-like construct forming seeding density for these cell types also, similar to those of dermal fibroblasts. These data indicate that a minimum and maximum cell seeding density per unit area exist for tissue formation by macromass culture. The range of high cell densities at which tissue formation occurs by macromass culture could be different for other cell types not tested.

The lower seeding densities of cells used as shown in Table 1 resulted in thinner tissue-like constructs, that is, having smaller three dimensions, while the higher seeding densities used resulted in thicker tissue-like constructs, that is, having larger three-dimensions. Thus, the dimensions of the three-dimensional tissue-like constructs can be varied.

TABLE 1
Dependence of three-dimensional tissue-like construct formation on
cell-seeding density.

| Total cells plated | Dermal | | |
|---|---|---|---|
| per cm² | fibroblasts | Adipose stromal cells | Chondrocytes |
| 1.3 × 10⁴ | − | − | − |
| 6.0 × 10⁴ | − | − | − |
| 1 × 10⁵ | − | − | − |
| 3.0 × 10⁵ | + | + | +/−(very weak) |
| 7 × 10⁵ | + | + | + |
| 1 × 10⁶ | + | + | + |
| 3 × 10⁶ | + | +/−(less cohesive) | + |
| 7 × 10⁶ | − | − | − |
| 10 × 10⁶ | − | − | − |

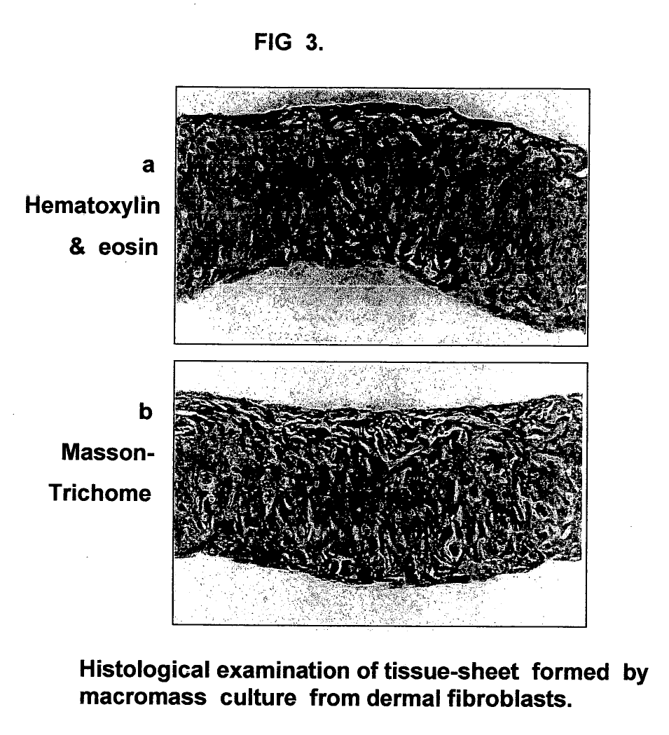

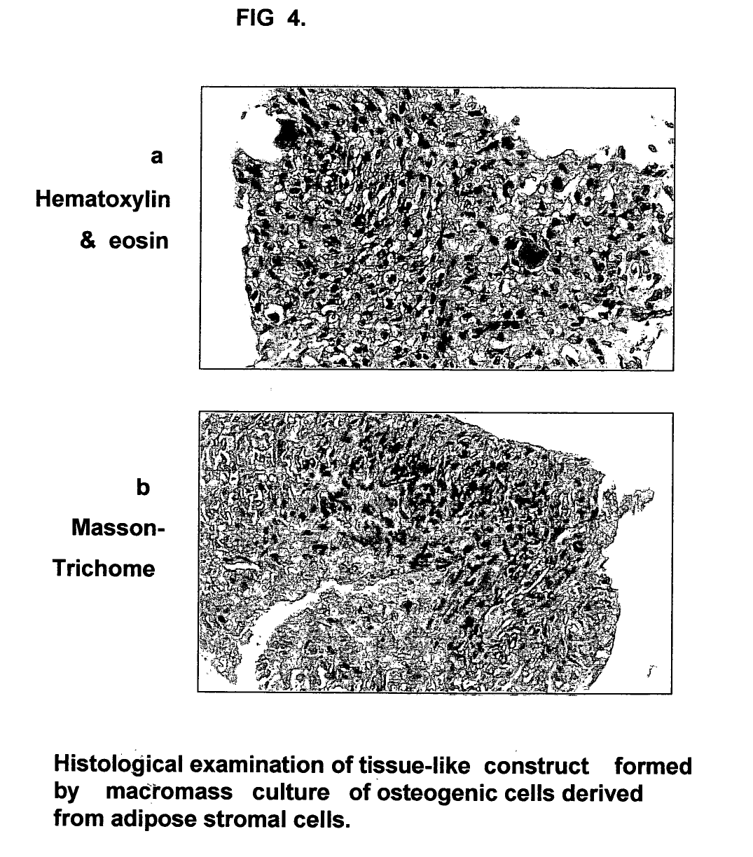

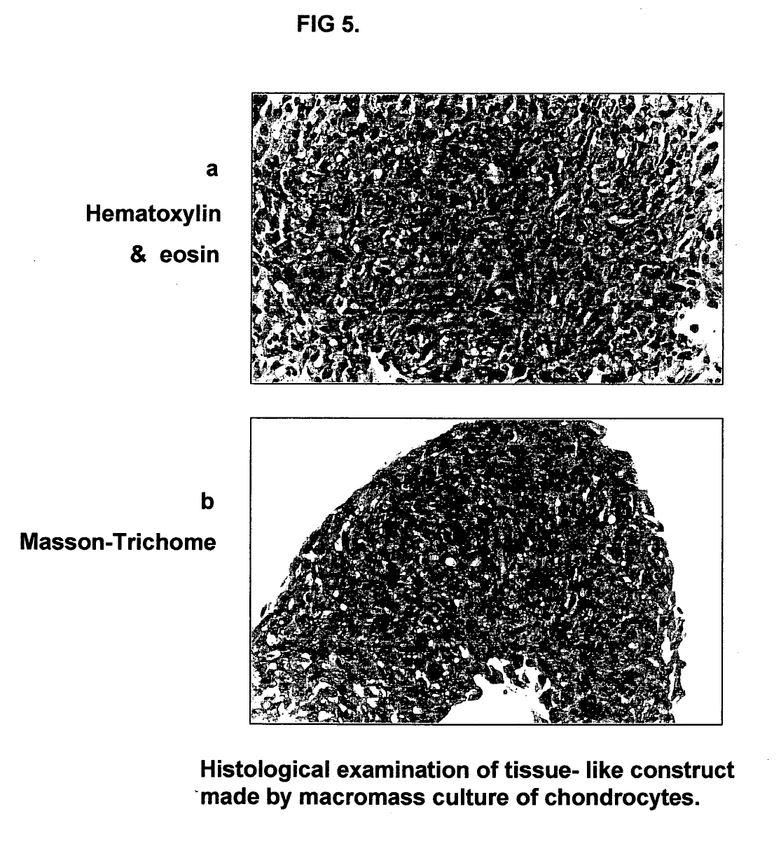

8.2. Histology (FIGS. 3, 4, 5)

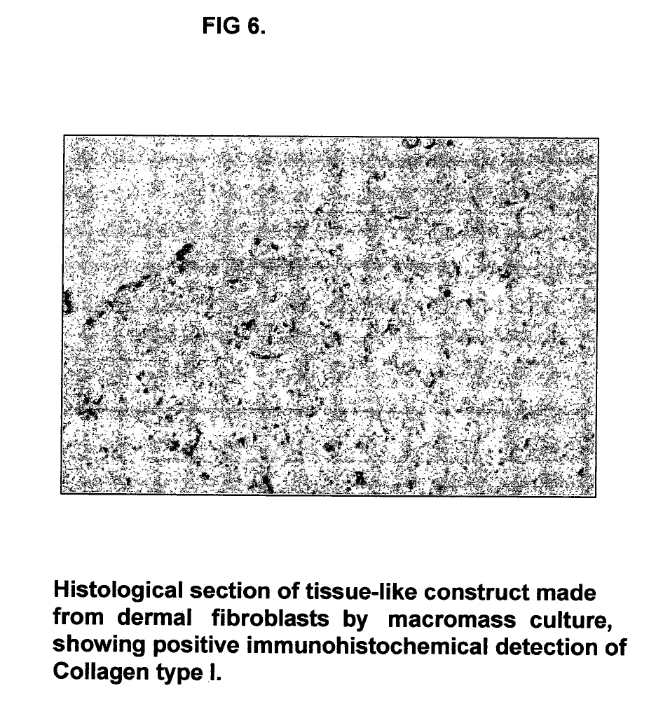

Hematoxylin& eosin staining of the sections of the different tissue-like constructs of the present invention shows three-dimensional structural organization and extracellular matrix formation. Masson-Trichome staining of tissue-like constructs formed from dermal fibroblasts as well as native stromal cells and osteogenic stromal cells showed collagen synthesis. Collagen type I immunostaining of a tissue-like construct made from dermal fibroblasts, incubated for 10 days, showed positive staining for collagen type I (FIG. 6). Thus, extracellular matrix formation appears to be taking place in tissue-like construct formation by the method of macromass culture.

8.3. Viability of Cells in Tissue-like Constructs Formed

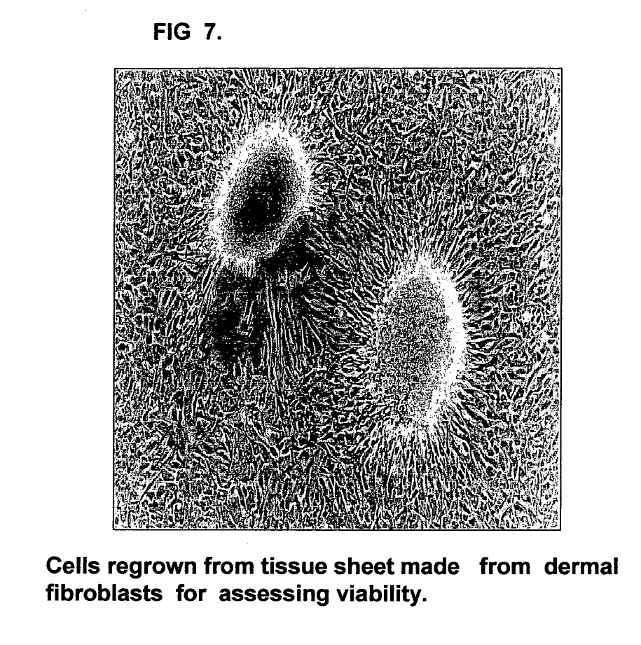

Cells re-isolated from the tissues formed from dermal fibroblasts and chondrocytes had viability greater than 98% by Trypan Blue staining; the cells from tissue mass from adipose stromal cells were about 90% viable. The isolated cells and clumps were plated to assess regrowth, and were found to be viable in each case, as depicted in FIG. 7 showing cells growing out of clumps of dissociated dermal fibroblast tissue sheet.

8.4. Expression Analysis of Tissue-like Constructs

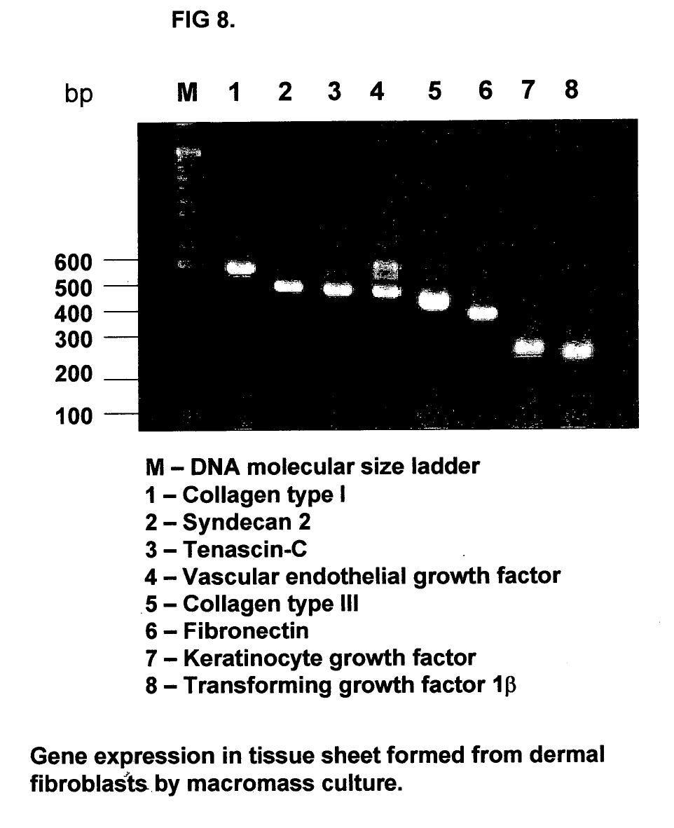

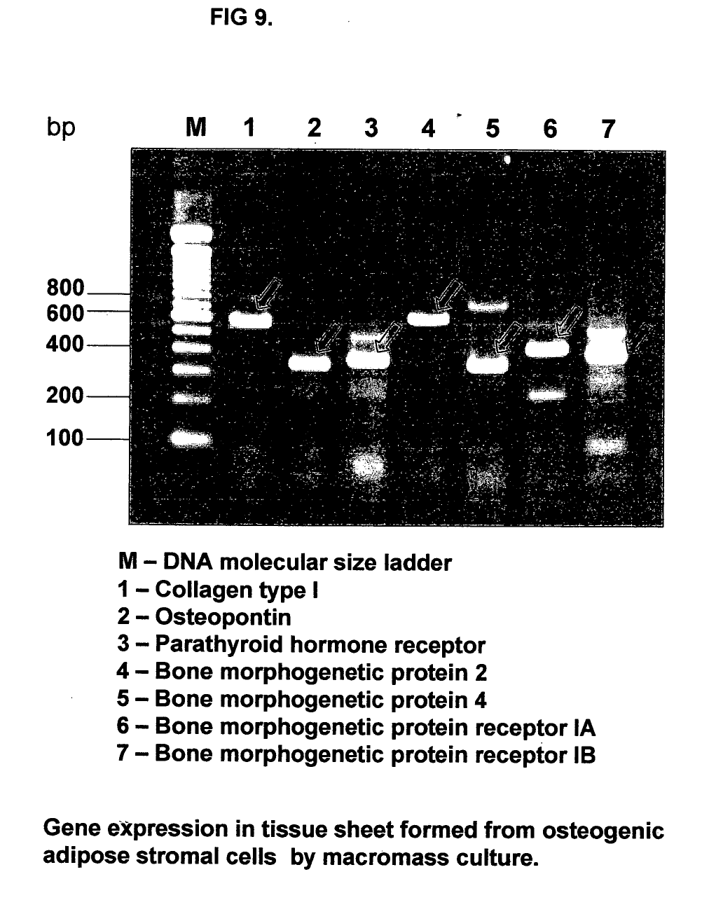

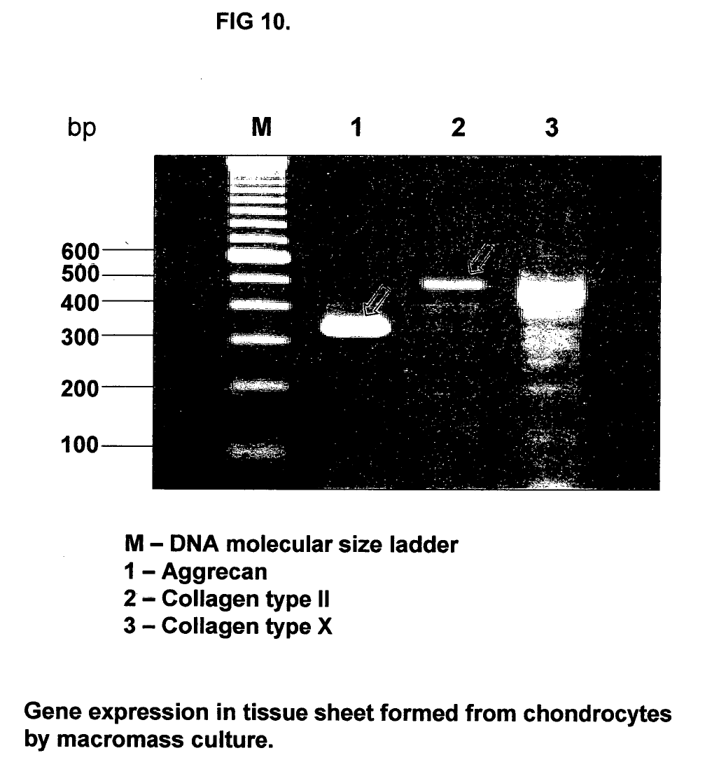

To assess whether the tissue sheet formed from dermal fibroblasts has potential as a dermis substitute, the expression of genes known to play an important role in the wound healing process of the skin was analyzed (FIG. 8). Collagen type I, collagen type III, keratinocyte growth factor, TGFβ1, fibronectin, vascular endothelial growth factor, tenascin-C and syndecan-2 were found to be expressed in the tissue sheet, thus demonstrating that this tissue-like sheet made from dermal fibroblasts has potential as a dermis substitute. To assess whether the tissue-like construct made from osteogenic adipose stromal cells had potential has a bone-like tissue substitute, the expression of bone-specific expressed genes was analyzed. The tissue-like construct was found to express collagen type I, osteopontin, parathyroid hormone receptor, bone morphogenetic protein 2, bone morphogenetic protein 4, bone morphogenetic protein receptors IA, IB & II, thus demonstrating its bone-like properties (FIG. 9), in addition to the focal calcification and actual bone spicule formation mentioned below. Similarly, the tissue-like construct made from chondrocytes was assessed for its potential as a cartilage tissue substitute. The cartilage-specific expressed genes collagen type II, aggrecan and collagen type X were found to be expressed, thus demonstrating its cartilage-like properties (FIG. 10), in addition to the formation of cartilage-specific extracellular matrix as mentioned below.

8.5 Modulation of Properties of Tissue-like Constructs by Flexibility of Tissue-formation Medium

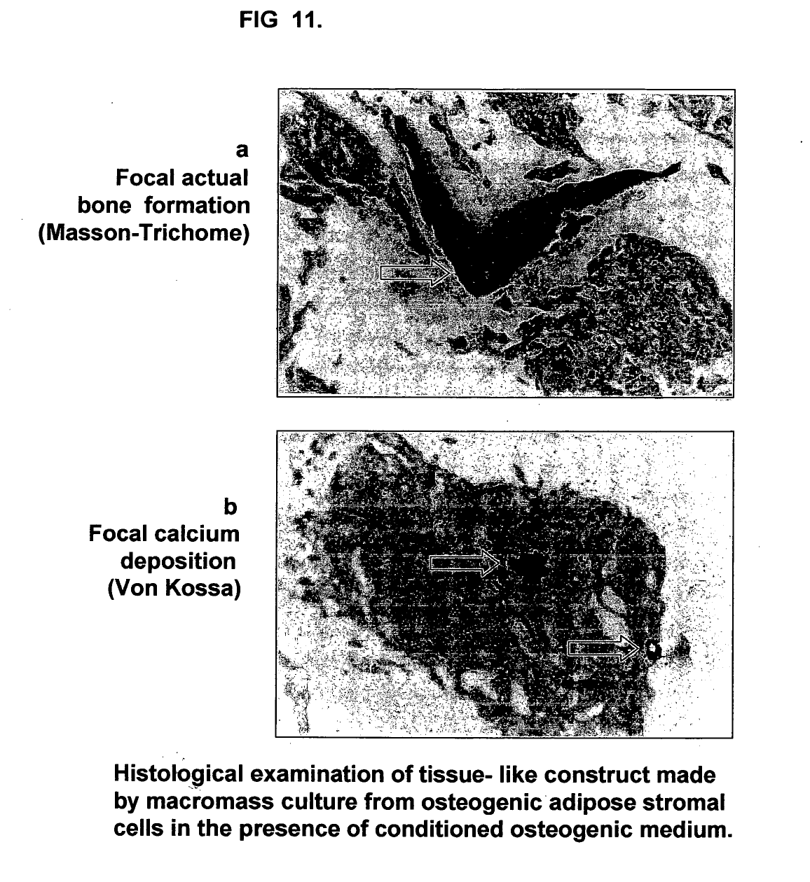

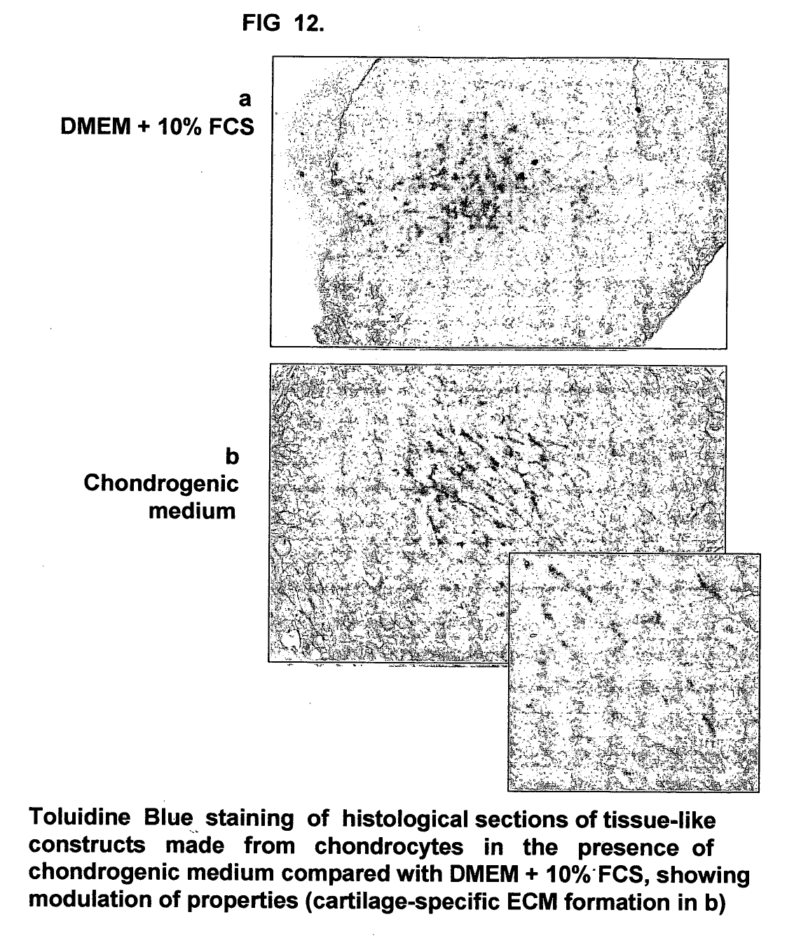

In macromass culture, it is possible to tailor the properties of the tissue formed by including appropriate medium components or by changing the medium, since, as far as has been tested, tissue formation is independent of medium conditions, as long as something that inhibits tissue-like organization by macromass is not included in the medium. This is apparent from tissue sheet formation by dermal fibroblasts in both serum-containing and serum free medium, as well as from tissue formation from adipose stromal cells in both DMEM+FCS and osteogenic medium, which contains dexamethasone and β-glycerophosphate. Thus, the properties of the tissue formed can be modulated to incorporate desirable properties by modifying the tissue-formation medium and/or growth medium of the cells. For instance, as presented in this invention, bone-like properties in the shape of actual bone formation were induced in tissue formed from adipose stromal cells by culturing the cells and forming the tissue-like construct in conditioned osteogenic medium as compared to osteogenic medium alone, where no actual bone spicule formation was seen (FIG. 11). Von Kossa staining of the tissue mass formed from osteogenic stromal cells in the presence of conditioned osteogenic medium showed focal regions of calcification, thus demonstrating bone-like properties, while the construct made from osteogenic stromal cells in non-conditioned osteogenic medium did not display these properties. Another example of such modulation is the formation of tissue-like constructs from chondrocytes in the presence of DMEM+10% FCS or in the presence of chondrogenic medium which contained 1% FCS and insulin; and TGFβ1 as a chondroinductive agent. The property of having extracellular matrix characteristic of the cartilage phenotype was generated in the tissue-like construct made in chondrogenic medium while the one made in DMEM+10% FCS did not have such a property. This is shown by the Toluidine blue staining in FIG. 12. Thus, even as it remains that tissue formation by macromass culture does not have specific complex medium requirements, such specific components can be used in the medium for modulating the properties of the tissue formed. It also follows from the above results that tissue-like organization and macroscopic tissue-like constructs can be formed in the presence of different culture media, with the different media presented here are examples and do not limit the invention hereof to these examples.

8.6. Growth Surface for Macromass Culture

The tissue-like sheets formed from cells plated on plastic surface had a tendency to detach spontaneously and curl or roll up, which is not desirable, since the sheet does not straighten once rolled up. A tissue being developed as a possible dermis substitute requires to remain straight. For this, macromass culture of dermal fibroblasts was done on a Hybond-N (Amersham Pharmacia Biotech, Buckinghamshire, UK) filter placed in a plastic dish. With this adaptation, the sheet that formed remained straightened and adhered to the filter, so, in the future, it could be applied onto a skin wound with the filter side up. This experiment demonstrates that it can be possible to achieve tissue formation by macromass culture on different compatible growth surfaces. Thus, in this case, the sheet made by macromass culture becomes a component of a putative implant that includes a nylon filter which serves as a supporting layer and its requirement is not as a scaffold for tissue-like organization of the cells. This supporting layer is not designed to integrate into the body along with the dermis-like sheet, and so it is not a scaffold which would become a part of the body, but a supporting handling device for the application of the dermis-like sheet. This supporting layer would be removed after healing.

8.7.Tissue-like Construct as a Component of an Object

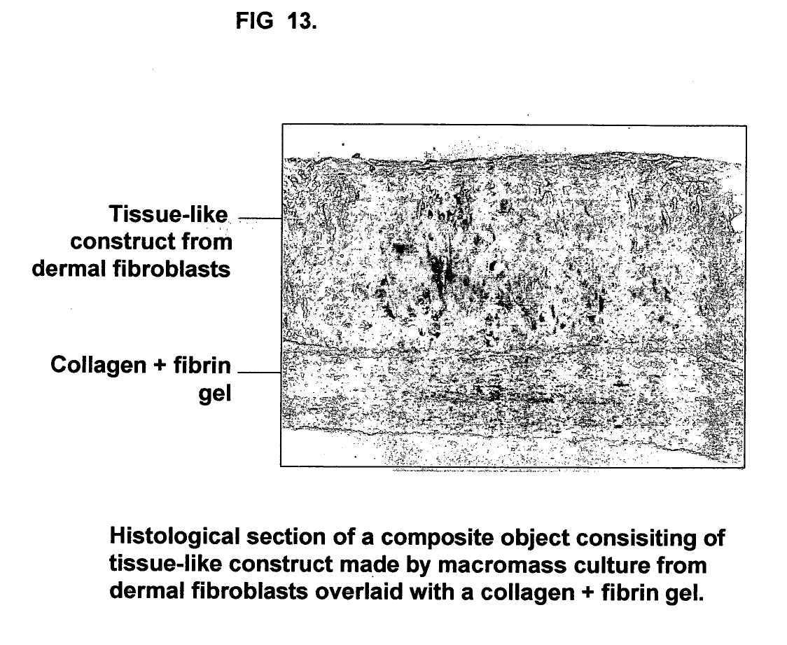

To demonstrate that a tissue-like construct can be incorporated to become a component or part of a (larger) object, dermal fibroblasts were seeded by macromass culture to form a tissue-like sheet, and then after removing the culture medium, it was overlaid with a collagen and fibrin gel mix. The gel was then allowed to set. Now, the gel could be lifted with the tissue-like sheet adhered to one side of it. A histological section of this composite, in which the tissue-like construct is a component, is shown in FIG. 13. Apart from gels and sheets, other matrices like sponges or membranes could also support tissue-like-constructs from one side by adhering to them. Thus, tissue-like organization and macroscopic tissue-like constructs can be combined with different objects, for example, a sheet or membrane, a gel, a sponge, or other matrices.

8.8. Microscopic Three-dimensional Tissue-like Organization by Macromass Culture

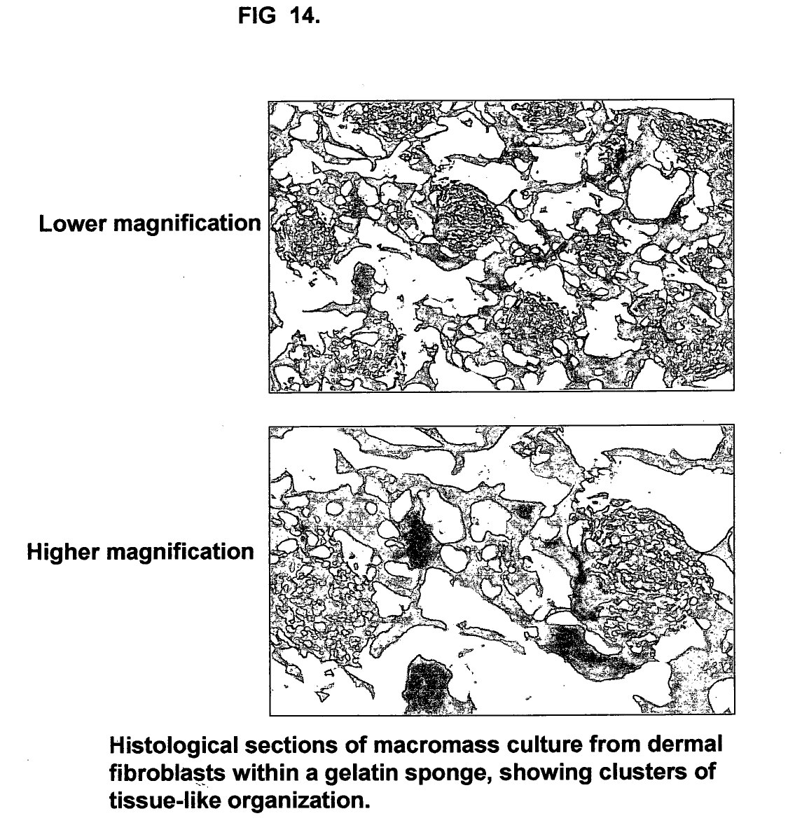

Dermal fibroblasts were seeded onto a gelatin sponge of diameter 3.5 cm, at a seeding density of about 2.5×10⁶ cells per cm² of sponge area, in DMEM+10% FCS. This application of the high cell-seeding-density macromass culture method to the gelatin sponge resulted in formation of microscopic clusters of three-dimensional tissue-like organizations within the sponge, as seen in the histological section of the sponge, shown in FIG. 14. Thus, the method of macromass culture can be used to generate three-dimensional tissue-like organization of cells at the microscopic level also, the tissue-like organizations becoming a component of the whole assembly.

### CONCLUDING REMARKS

The novelty of the present invention lies in the fact that high cell-seeding-density culture can generate whole three-dimensional tissue-like organization of cells without any specific agents that aid in tissue formation to induce such organization and can be scaled up areawise to generate macroscopic three-dimensional tissue-like constructs of different kinds, besides in the other important features such as the fact that scaffolding material is not employed or specific agents that aid in tissue formation/complex media formulations are not required, as are detailed in this description. The inventors of the present invention are the first to report that whole three-dimensional tissue-like organization of cells and macroscopic three-dimensional tissue-like constructs of different kinds can be generated by high cell-seeding-density culture alone.

It may be possible that other mesodermal cells or cells of endodermal or ectodermal origin can also form such tissue-like organization by macromass culture. Hence, tissue-like organization and macroscopic tissue-like constructs made by macromass culture from any type of mammalian cells, if they are possible, are within the scope of this invention, the defining feature being tissue-like organization achieved by the method of macromass culture.

While macroscopic tissue-like constructs are the preferred embodiment of this invention, it is apparent that macromass culture at a smaller scale, that is in culture areas anything smaller than about 0.75 cm diameter, would result in smaller tissue-like organizations, decreasing in size towards being microscopic as the scale of macromass culture is reduced. Microscopic tissue-like organization by high cell-seeding-density macromass culture in the above form or other form such as in the gelatin sponge described earlier is thus also within the scope of this invention. Likewise, tissue-like constructs could be generated by macromass culture in culture vessels larger than 8.5 cm diameter.

Thus, in the foregoing description, specific embodiments of this invention have been described: examples have been presented with respect to the aspects of use of different cell types, the use of alternative growth surface, the use of change in medium to modulate the properties of the tissue formed, the scale-up of tissue formation, the sizes of culture vessels, the range of tissue-forming high cell densities, and the generation of a putative implant of which tissue made by macromass culture is a component. Although, only the described embodiments have been brought forth, they serve the purpose of example or illustration only, and do not limit the invention.

It should remain understood that different modifications or substitutions could be made to this invention, which would be within the scope of the present invention. For instance, it is contemplated by the inventors that one such modification would be the entrapment or encapsulation of tissue masses made by macromass culture into a suitable gel or matrix, or another such modification would be a construct in which multiple tissue-like masses or sheets are joined or held together by some means. In such constructs, the tissue-like construct made by macromass culture would now be a component of the whole substitute. The above are other ways, than those presented in the Results, by which tissue-like organization and constructs generated by macromass culture can be a component of an object. As demonstrated in the Results, even as it remains that tissue-like organization of cells by macromass culture does not require any scaffold and also histologically competent tissue-like constructs form without the aid of a scaffold, a scaffold may be provided for macromass culture, for example, as an alternative growth surface, such that the scaffold becomes a part of the whole substitute. Thus, the three-dimensional tissue-like organization and histologically competent tissue-like constructs of this invention can be developed scaffold-free, but can be combined with a suitable scaffold, if it gives beneficial properties or advantages over the tissue-like construct alone. Other modifications would be the use of other cell types which have the properties to form a tissue-like mass by macromass culture, use of other compatible growth surface than described, other medium changes for modulation, and other sizes of scale-up, etc. Therefore, this description of the present invention is not intended to limit this invention by the precise illustrative embodiments that are disclosed.

### REFERENCES CITED

U.S. PATENT DOCUMENTS

| | | |
|---|---|---|
| 5489304 | February 1996 | Orgill, et al. |
| 5460939 | October 1995 | Hansbrough, et al. |
| 5613982 | March 1997 | Goldstein. |
| 20020127711 | September 2002 | Kale, et al. |
| 5723331 | March 1998 | Tubo, et al. |

### OTHER REFERENCES

Abu-Absi S F, Friend J R, Hansen L K, Hu W (2002) Structural polarity and functional bile canaliculi in rat hepatocyte spheroids. Experimental Cell Research 274, 56-67.

Ahrens P B, Solursh M, Reiter R S (1977) Stage-related capacity for limb chondrogenesis in cell culture. Developmental Biology 60, 69-82.

Baldwin S P, Saltzman W M (2001) Aggregation enhances catecholamine secretion in cultured cells. Tissue Engineering 7(2)179-190.

Bancroft J D, Cook H C (1994) Manual of Histological Techniques and their Diagnostic Application, Churchill Livingstone, Edinburgh, UK.

Buttery L D K, Bourne S, Xynos J D, Wood H, Hughes F J, Hughes S P F, Episkopou V, Polak J M (2001) Differentiation of osteoblasts and in vitro bone formation from murine embryonic stem cells. Tissue Engineering 7(1)89-99.

Davies CdeL, Berk D A, Pluen A, Jain R K (2002) Comparison of IgG diffusion and extracellular matrix composition in rhabdomyosarcomas grown in mice versus in vitro as spheroids reveals the role of host stromal cells. British Journal of Cancer 86, 1639-1644.

Denker A E, Nicoll S B, Tuan R S (1995) Formation of cartilage-like spheroids by micromass cultures of murine C3H10T1/2 cells upon treatment with transforming growth factor-beta 1. Differentiation 59(1)25-34.

Downie S A, Newman S A (1994) Morphogenetic differences between fore and hind limb precartilage mesenchyme: Relation to mechanisms of skeletal pattern formation. Developmental Biology 162, 195-208.

Eaglstein W H, Falanga V (1997) Tissue engineering and the development of Apligraf®, a human skin equivalent. Clinical Therapeutics 19(5)894-905.

Furukawa K S, Ushida T, Sakai Y, Suzuki M, Tanaka J, Tateishi T (2001) Formation of human fibroblast aggregates (spheroids) by rotational culture. Cell Transplantation 10, 441-445.

Hamamoto R, Yamada K, Kamihira M, Iijima S (1998) Differentiation and proliferation of primary rat hepatocytes cultured as spheroids. Journal of Biochemistry 124, 972-979.

Johnstone B, Hering T M, Caplan A I, Goldberg V M, Yoo J U (1998) In vitro chondrogenesis of bone marrow-derived mesenchymal progenitor cells. Experimental Cell Research 238, 265-272.

Kaji E H, Leiden J M (2001) Gene and stem cell therapies. Journal of the American Medical Association 285(5)545-550.

Kale S, Biermann S, Edwards C, Tarnowski C, Morris M, Long M W (2000) Three-dimensional cellular development is essential for ex vivo formation of human bone. Nature Biotechnology 18, 954-958.

Kim H W, Han C D (2000) An overview of cartilage tissue engineering. Yonsei Medical Journal 41(6)766-773.

Lang S H, Stark M, Collins A, Paul A B, Stower M J, Maitland N J (2001) Experimental prostate epithelial morphogenesis in response to stroma and three-dimensional Matrigel culture. Cell Growth & Differentiation 12, 631-640.

L'Heureux N, Pâquet S, Labbé R, Germain L, Auger F A (1998) A completely biological tissue-engineered human blood vessel. FASEB Journal 12, 47-56.

Mackay A M, Beck S C, Murphy J M, Barry F P, Chichester C O, Pittenger M F (1998) Chondrogenic differentiation of cultured human mesenchymal stem cells from marrow. Tissue Engineering 4(4)415-428.

McPherson J M, Tubo R (2000) Articular cartilage injury. In Principles of Tissue Engineering, 2^{nd} Edition, Academic Press, San Diego, USA, p.697-709.

Michel M, L'Heureux N, Pouliot R, Xu W, Auger F A, Germain L (1999) Characterization of a new tissue-engineered human skin equivalent with hair. In vitro Cellular & Developmental Biology—Animal 35, 318-326.

Pollok J M, Kluth D, Cusick R A, Lee H, Utsunomiya H, Ma P X, Langer R, Broelsch C E, Vacanti J P (1998) Formation of spheroidal aggregates of hepatocytes on biodegradable polymers under continuous flow bioreactor conditions. Journal of Pediatric Surgery 8, 195-199.

Service R F (2000) Tissue engineers build new bone. Science 289, 1498-1500.

Shinji T, Koide N, Tsuji T (1988) Glycosaminoglycans partially substitute for proteoglycans in spheroid formation of adult rat hepatocytes in primary culture. Cell Structure & Function 13, 179-188.

Takezawa T, Mori Y, Yonaha T, Yoshizato K (1993) Characterization of morphology and cellular metabolism during the spheroid formation by fibroblasts. Experimental Cell Research 208, 430-441.

Yamada K, Kamihira M, Hamamoto R, Iijima S (1998) Efficient induction of hepatocyte spheroids in a suspension culture using a water-soluble synthetic polymer as an artificial matrix. Journal of Biochemistry 123, 1017-1023.

Yoon Y, Oh C, Kim D, Lee Y, Park J, Huh T, Kang S, Chun J (2000) Epidermal growth factor negatively regulates chondrogenesis of mesenchymal cells by modulating the protein kinase C-α, Erk-1, and p38 MAPK signaling pathways. Journal of Biological Chemistry 275(16)12353-12359.

Zuk P A, Zhu M, Mizuno H, Huang J, Futrell J W, Katz A J, Benhaim P, Lorenz H P, Hedrick M H (2001) Multilineage cells from human adipose tissue: implications for cell-based therapies. Tissue Engineering 7(2)211-228.

1. A method for the generation of living tissue-like organization of cells, viz., macromass culture, including three-dimensional tissue-like constructs, free from the requirement of scaffold or extraneous matrix, comprising:
a culture system in which cells are seeded at a high density per unit area of a culture vessel in a range spanning a window around 10⁶ cells per cm² resulting in three-dimensional tissue-like formation or organization of cells, free from the requirement for any other agents that aid in tissue formation; and
providing tissue like constructs made from mesodermal cells, and could be applicable to other cell types;

2. The method as claimed in claim 1, including using macromass culture comprising a culture system for tissue formation, which comprises:
generating three-dimensional tissue-like organization, macroscopic or microscopic, from cells by high-density cell seeding; and
bringing the cells together in close proximity in a certain favorable range of high densities of cells in three-dimensional space, that favors cohesive integration of cells into a three-dimensional tissue-like state, free of the requirement for any other agents that aid in tissue formation.

3. Tissue-like organizations of cells including macroscopic three-dimensional constructs according to claim 1 for use as tissue substitutes for implantation, for wound healing, as in vitro models for drug testing, and the like, made from fibroblastic cells of mesenchymal origin, including:
engineering a putative dermal equivalent made from dermal fibroblasts, putative substitute with bone-like properties made from osteogenic cells derived from adipose stromal cells, and putative cartilage substitute made from chondrocytes, but not necessarily limited to these cell types; and
generating tissue-like organizations of cells which can be made to assume different forms.

4. Tissue-like organizations of cells as claimed in claim 3, which are three-dimensional in nature and encompasses three-dimensional macroscopic tissue-like constructs.

5. Tissue-like organizations of cells as claimed in claim 3, which can be made to assume different forms, being generated for the purpose of achieving different properties or qualities, said different forms comprising:
three-dimensional macroscopic tissue-like constructs by themselves, wherein “macroscopic” means that the size of the tissue is at least such that it can be easily visually discerned by normal human vision, and the macroscopic tissue-like constructs are histologically competent; and
combining the three-dimensional tissue-like organization with different matrices, such as gels, sheets, membranes or sponges or with other scaffolds and the like; and
said tissue-like organization being in the form of microscopic three-dimensional structures.

6. Tissue-like organizations of cells as claimed in claim 3, including tissue-like organizations which can be generated wholly using cells alone and a culture medium.

7. Tissue-like organizations of cells as claimed in claim 3, wherein the tissue-like organization of cells can be generated without using any agents that aid in tissue-formation, comprising:
tissue-inducing chemicals such as ascorbic acid;
tissue-inducing growth factors; and
substratum with special properties; rotational culture; complex bioreactor; or extraneous scaffold or matrix or supports.

8. Tissue-like organizations of cells as claimed in claim 3, including tissue-like organizations which can be generated free of the requirement for extraneous extracellular matrix components.

9. Tissue-like organizations of cells as claimed in claim 3, generated from different cell types comprising:
dermal fibroblasts;
adipose stromal cells;
osteogenic cells derived from adipose stromal cells;
chondrocytes; and
osteoblasts.

10. Tissue-like organizations of cells produced by macromass culture as claimed in claim 3, for the formation of which a range of cell seeding densities exists that favors tissue formation and the exact range of tissue-forming high cell seeding densities are possible to be different for different cell types.

11. Tissue-like organizations of cells by macromass culture as claimed in claim 3, wherein the time of formation can vary for different cell types or different media conditions.

12. Tissue-like organizations of cells produced by macromass culture as claimed in claim 3, generated free of the requirement for specific media conditions, including:
serum-free media conditions; and
specific complex media compositions.

13. Tissue-like organizations of cells produced by macromass culture and the different forms thereof as claimed in claim 3, that are three-dimensional and that have flexibility with respect to dimensions comprising:
different three dimensions sizes;
larger or smaller tissue-like constructs by scaling up or down macromass culture; and
variable size or scale by changing the number of cells used to achieve a seeding density within a macromass favorable range of tissue-forming densities by scaling up or down the macromass culture.

14. Tissue-like organizations of cells as claimed in claim 3, having a flexibility with respect to culture media used, comprising:
formation of the tissue-like organization in the presence of different culture media, both, different growth and/or tissue-formation media; and
modulating the properties of the tissue-like organization by including components in the growth and/or tissue-formation medium, provided that addition of these components does not adversely affect tissue formation, or by changing the medium, provided that this change in medium does not adversely affect tissue formation.

15. Tissue-like organizations of cells by macromass culture as claimed in claim 3, wherein the tissue substitutes are achieved on different compatible growth surfaces or scaffolds.

16. Tissue-like organization as claimed in claim 3, including the preparation of the tissue-like constructs made in culture vessels of any shape, with a flat or curved base.

17. Tissue-like organizations of cells as claimed in claim 3, which can be made to assume different forms, and different forms being generated for the purpose of achieving different properties or qualities, comprising:
three-dimensional macroscopic tissue-like constructs having a size that can be easily visually discerned by normal human vision;
the macroscopic tissue-like constructs being histologically competent;
combining the three-dimensional tissue-like organization with different matrices, such as gels, sheets, membranes or sponges or with other scaffolds; and
the tissue-like organization of cells being in the form of microscopic three-dimensional structures.

18. A method for the generation of tissue-like organization of cells including fabrication of three-dimensional tissue-like constructs free of the aid of scaffold comprising:
employing high cell-seeding-density culture to generate tissue-like organization of cells free of the requirement for employing specific agents that aid in tissue formation and scaffolds;
providing tissue-like constructs made from mesodermal cells, but not necessarily limited to these cell types; and
constructing the tissue-like organization of cells to produce different tissue engineered products by generating tissue-like organization of cells and formation of living, cellular putative tissue substitutes.

19. The method as claimed in claim 18, including using high cell seeding density per unit area or space of culture vessel free of the requirement for other agents to form the tissue-like organization of cells and to provide macroscopic tissue-like constructs.

20. The method as claimed in claim 18, including formation of tissue-like organization using macromass culture by seeding the cells at a high cell density per unit area or space of culture vessel.

21. The method as claimed in claim 20, wherein the macromass culture comprises a culture system for tissue formation, comprising:
seeding cells at a high density per unit area or space of the culture vessel in a range spanning a window around 10⁶ cells per cm² and free of the requirement for other agents that aid in tissue formation; and
the macromass culture further comprising:
generating tissue-like organization, macroscopic or microscopic, from cells by high-density cell seeding, bringing cells together in close proximity in a certain favorable range of high densities of cells in three-dimensional space, free of the requirement for any other agents that aid in tissue formation;
achieving the macromass range of favorable high cell seeding densities by settling the cells together within the three-dimensional space occupied by the cells at the base of the culture vessel such that they come into a state of close proximity with one another that triggers or signals them into a tissue formation mode by which they become cohesively integrated; and
achieving the macromass range of cell seeding density in a vessel with a flat or curved base whereby using a culture vessel of at least about 0.75 cm in diameter for macromass culture results in the formation of macroscopic tissue-like constructs, and macroscopic defines a tissue size that can be easily visually discerned by the normal human vision.

22. Tissue-like constructs for implantation in a human or mammalian body, made in accordance with a method for generating macroscopic tissue-like constructs and whole tissue-like organization of cells free of the requirement for any specific agents to induce organization and can be scaled up to generate macroscopic tissue-like constructs free of the requirement for scaffolding material and specific agents/complex media formulations, solely by high cell-seeding-density culture, viz., macromass culture from cells comprising those of mesenchymal origin, wherein the tissue-like organization of cells and putative tissue equivalents made, are made from cells of mesenchymal origin including an engineered putative dermal equivalent made from dermal fibroblasts, putative substitute with bone-like properties made from adipose stromal cells-derived osteogenic cells or from osteoblasts and putative substitute for cartilage repair made from chondrocytes.

23. The tissue-like constructs as claimed in claim 22, wherein:
the tissue-like organization of cells and macroscopic tissue like constructs are made without the requirement for scaffold or extraneous matrix or complex bioreactor for tissue generation to produce three-dimensional tissue-like constructs for implantation in a human or mammalian body as therapy for diseased or damaged conditions;
formation of the tissue-like constructs is free of the requirement for a pre-shaped well having a surface detrimental for cell attachment; and
the tissue-like organization of cells and macroscopic tissue-like constructs are made free of the requirement for any agents such as tissue-inducing chemicals, tissue-inducing growth factors, substratum with special properties, rotational culture.
